(19) United States
(12) Patent Application Publication
(10) Pub. No.: US 2006/0083770 A1
(43) Pub. Date: Apr. 20, 2006

(54) Medical devices and methods of preparation and use

(75) Inventors: Andreas Greiner, Amoneburg (DE); Phillip Hanefeld, Klein Parin (DE); Rakesh Kumar, Carmel, IN (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Specialty Coating Systems, Inc., Indianapolis, IN (US)

(21) Appl. No.: 10/966,985

(22) Filed: Oct. 15, 2004

(51) Int. Cl. A61K 6/083; A61F 2/00; B05D 3/00

(52) U.S. Cl. 424/426; 427/2.26

(57) ABSTRACT

An implantable device having a coating with an eluting membrane and a permeable membrane disposed on a surface of the device is provided. The eluting membrane is typically a polymeric material capable of being infused with a biologically active, therapeutic substance. The permeable membrane is typically a polymeric material having a thickness that controls the rate of transport of the biologically active material to a target tissue. The polymeric materials are typically parylene or derivatives thereof.

### BACKGROUND OF THE INVENTION

1. Field of Invention

The present invention relates to medical devices as well as to methods of preparation and use thereof and, in particular, to implantable medical devices, such as stents, having drug-eluting coatings and methods of preparation and use of such medical devices.

2. Discussion of Related Art

Devices, including implantable medical devices, having coatings deposited on a surface thereof have been disclosed. For example, Fearnot et al., in U.S. Pat. No. 5,609,629, disclosed a coated implantable medical device. Ding et al., in U.S. Pat. No. 6,358,556, disclosed a drug release stent coating. Ding et al., in U.S. Pat. No. 6,364,856, also disclosed a medical device with sponge coating for controlled drug release. Ding, in U.S. Pat. No. 6,249,952, disclosed a method for manufacturing an expandable stent. Harish et al., in U.S. Pat. No. 6,506,437, disclosed methods of coating an implantable device having depots formed in a surface thereof. Ragheb et al., in U.S. Pat. Nos. 5,824,049, 6,096,070, and 6,299,604, disclosed a coated implantable medical device. Ragheb et al., in U.S. Pat. No. 5,873,904, also disclosed a silver implantable medical device. Dinh et al., in U.S. Pat. Nos. 5,591,227 and 5,697,967, disclosed a drug eluting stent. Dinh et al., in U.S. Pat. No. 5,599,352, also disclosed a method of making a drug eluting stent and, in U.S. Pat. No. 6,399,144, disclosed a medical device for delivering a therapeutic substance and method therefor. Boatman et al., in U.S. Pat. Nos. 5,632,771 and 6,409,752, disclosed a flexible stent having a pattern formed on a sheet of material. Boatman et al., in U.S. Pat. No. 6,464,720, also disclosed a radially expandable stent. Tuch, in U.S. Pat. Nos. 5,679,400 and 5,776,184, disclosed an intravascular stent and method. Tuch, in U.S. Pat. No. 5,824,048, also disclosed a method for delivering a therapeutic substance to a body lumen. Tartaglia et al., in U.S. Pat. Nos. 5,637,113 and 5,700,286, disclosed a polymer fill for wrapping a stent structure. Berg et al., in U.S. Pat. No. 5,837,008, disclosed an intravascular stent and method. Yan, in U.S. Pat. No. 5,843,172, disclosed a porous medicated stent. Leone et al., in U.S. Pat. No. 5,882,335, disclosed a retrievable drug delivery stent; in U.S. Pat. No. 5,891,108, disclosed a drug delivery stent; and, in U.S. Pat. No. 5,902,266, disclosed a method for delivering a liquid solution to the interior wall surface of a vessel. Loeffler, in U.S. Pat. No. 5,897,911, disclosed a polymer-coated stent structure. Steinke et al., in U.S. Pat. No. 6,033,436, disclosed an expandable stent. Callol et al., in U.S. Pat. No. 6,174,329, disclosed a protective coating for a stent with intermediate radiopaque coating. Tedeschi et al., in U.S. Pat. No. 6,218,016, disclosed a lubricious, drug-accommodating coating. Berry et al., in U.S. Pat. No. 6,231,598, disclosed a radially expandable stent. Wu et al., in U.S. Pat. No. 6,254,632, disclosed an implantable medical device having protruding surface structures for drug delivery and cover attachment. Michal et al., in U.S. Pat. No. 6,287,285, disclosed a therapeutic, diagnostic, or hydrophilic coating for an intracorporeal medical device. Wang et al., in U.S. Pat. No. 6,331,186, disclosed an end sleeve coating for stent delivery. Chudzik et al., in U.S. Pat. No. 6,344,035, disclosed a bioactive released coating. Bashiri et al., in U.S. Pat. No. 6,468,266, disclosed a fast detaching electrically isolated implant. Bates et al., in U.S. Pat. No. 6,530,951, disclosed a silver implantable medical device. Yoe, in U.S. Pat. No. 6,544,582, disclosed a method and apparatus for coating an implantable device. Barry, in U.S. Patent Application Publication No. 2002/0077592, disclosed a replenishable stent and delivery system.

Khair et al., in U.S. Pat. No. 5,425,710, disclosed a coated sleeve for wrapping dilatation catheter balloons. Tsukernik et al., in U.S. Pat. No. 5,868,719, disclosed a drug delivery balloon catheter device.

Further, Nicholas et al., in U.S. Pat. No. 5,588,962, disclosed drug treatment of diseased sites deep within the body. Eury et al., in U.S. Pat. No. 5,605,696, disclosed a drug loaded polymeric material and method of manufacture. Williams, in U.S. Pat. No. 5,707,385, disclosed a drug loaded elastic membrane and method for delivery. Sahatjian et al., in U.S. Pat. Nos. 5,674,192 and 5,954,706, disclosed drug delivery. Grantz, in U.S. Pat. No. 6,129,705, disclosed a drug delivery and gene therapy delivery system. Myers et al., in U.S. Pat. Nos. 6,159,978, 6,180,632, and 6,528,526, disclosed quinoline and quinoxaline compounds which inhibit platelet-derived growth factor and/or P56LCK tyrosine kinases. He et al., in U.S. Pat. No. 6,245,760, and Spada et al., in U.S. Pat. No. 6,482,834, also disclosed quinoline and quinoxaline compounds which inhibit platelet-derived growth factor and/or P56LCK tyrosine kinases. Tam et al., in U.S. Pat. No. 6,261,320, disclosed a radioactive vascular liner. Kamath et al., in U.S. Pat. No. 6,335,029, disclosed polymeric coatings for controlled delivery of active agents. Chudzik et al., in U.S. Pat. No. 6,214,901, disclosed a bioactive agent release coating. Schwarz et al., in U.S. Pat. No. 6,368,658, disclosed coating medical devices using air suspension.

Hunter et al., in U.S. Pat. No. 5,886,026, disclosed anti-angiogenic compositions and methods of use. Anderson et al., in U.S. Pat. No. 6,254,634, disclosed coating compositions. End et al., in U.S. Pat. No. 6,365,600, disclosed imidazol methyl quinolinone derivatives and inhibitors of smooth muscle cell proliferation. Sirhan et al., in U.S. Pat. No. 6,471,980, disclosed intravascular delivery of mycophenolic acid.

However efforts aimed at addressing the deficiencies of the related art can advantageously provide benefits including, for example, providing techniques that allow disposition of one or more elutable substances on a device and/or disposition of one or more materials that controllably regulate the release of the elutable substance.

### SUMMARY OF THE INVENTION

In accordance with one or more embodiments, the invention is directed to a method of coating a medical device. The method can comprise depositing an eluting membrane on a surface of the medical device and depositing a barrier membrane comprising a permeable polymer on the eluting membrane. Preferably, the eluting membrane comprises a non-bioerodible polymer having a biologically active material dispersed therein.

In accordance with one or more embodiments, the invention is directed to a method of coating an implantable medical device. The method can comprise depositing a first polymer matrix comprising parylene or a derivative thereof on a surface of the implantable medical device, infusing the first polymer matrix with a therapeutic agent, and depositing a second polymer matrix comprising parylene or a derivative thereof on the first polymer matrix.

In accordance with one or more embodiments, the invention is directed to a method of administering a drug to a patient. The method can comprise providing a medical device comprising a drug-eluting membrane, disposed on a surface of the medical device, and a permeable membrane, disposed on the drug-eluting membrane. The method further comprises implanting the medical device in the patient. Preferably, the drug-eluting membrane comprises a non-bioerodible polymer and a drug infused therein. More preferably, the permeable membrane comprises a polymer.

In accordance with one or more embodiments, the invention is directed to a method of coating a medical device. The method can comprise depositing a first polymer matrix comprising parylene or a derivative thereof on a surface of the medical device and infusing the first polymer matrix with a therapeutic agent.

In accordance with one or more embodiments, the invention is directed to a method of facilitating delivery of a therapeutic agent. The method comprises providing the medical device comprising an eluting membrane disposed on a surface of the medical device and a permeable membrane disposed on the eluting membrane. Preferably, the eluting membrane comprises a therapeutic agent infused in a non-bioerodible polymer. More preferably, the permeable membrane comprises a polymer.

In accordance with one or more embodiments, the invention is directed to an implantable medical device. The implantable medical device comprises a body having a surface, a drug-eluting membrane disposed on the surface, and a permeable membrane disposed on the drug-eluting membrane. Preferably, the drug-eluting membrane comprises a therapeutic agent infused in a non-bioerodible polymer and the permeable membrane comprising a polymer.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic drawing of an implantable medical device in accordance with one or more embodiments of the invention;

FIG. 2 is a graph, in accordance with one or more embodiments of the invention, comparing the release of dexamethasone from parylene C membranes of different masses (18 mg, 28 mg, and 91 mg), but substantially the same surface area (about 37.5 mm²), loaded by immersion for about one day in a solution of about 20 ml CHCl₃ and about 15 mg dexamethasone; and

FIG. 3 is a graph, in accordance with one or more embodiments of the invention, showing the effect of dexamethasone drug loading time (immersion for about 1, 2, 3, 6, and 22 days, marked as “Tage”) of parylene C membranes of substantially the same mass (about 90 mg) and substantially about the same surface area (about 37.5 mm²);

FIG. 4 is a graph, in accordance with one or more embodiments of the invention, showing a comparison of the release of dexamethasone from an uncoated dexamethasone-loaded parylene N membrane (about 29 mg parylene N, surface area of about 37.5 mm², loading period of about one day) (-▪-), and a parylene N coated (about 800 nm membrane thick) dexamethasone-loaded parylene N membrane (about 32 mg parylene N, surface area of about 37.5 mm², loading period of about one day) (-●-); and

FIG. 5 is a graph, in accordance with one or more embodiments of the invention, showing a comparison of the release of dexamethsone from an uncoated dexamethasone-loaded parylene N membrane (about 18 mg parylene N, surface area about of 37.5 mm², loading period of about one day) (-●-), and a parylene N coated (about 1700 nm membrane thick) dexamethasone-loaded parylene N membrane (about 18 mg parylene N, surface area of about 37.5 mm², loading period of about one day) (-▪-).

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

In accordance with one or more embodiments, the present invention can provide a medical device comprising one or more surfaces that is at least partially coated with one or more membranes exhibiting eluting properties.

In accordance with one or more embodiments, the invention can comprise an article or device comprising one or more sources or reservoirs of one or more mobile agents or substances and one or more transport rate-controlling components. The source or reservoir typically contains or otherwise stores the agent. The source or reservoir can provide, deliver, or otherwise make available, one or more transportable, deliverable, or otherwise motile, substances or agents through, for example, the rate-controlling component. In some cases, the reservoir contains or otherwise makes available the one or more substances or agents in a, for example, controlled or uniform rate or in a total dosage or exposure amount. The rate-controlling component typically controls the transport rate of the one or more agents from the source. In accordance with one or more embodiments, the invention can be further characterized as a method of coating a structure, such as a device that can be implanted into a patient. In accordance with further embodiments, the invention can also be characterized as being directed to a method of coating an implantable medical device. The method can comprise an act of depositing a substance-eluting membrane on at least a portion of a surface of a device. The method can comprise acts of depositing a first polymer matrix comprising, for example, parylene or a derivative thereof, on at least a portion of the surface of the implantable medical device and disposing by, for example, infusing, the first polymer matrix with one or more therapeutic agents. The method can further comprise depositing a second polymer matrix comprising, for example, parylene or a derivative thereof, on at least a portion of the first polymer matrix. In accordance with one or more embodiments, the invention can be characterized as providing a method of administering a therapeutic agent to a patient. The method can comprise acts of providing an implantable device comprising a drug-eluting membrane disposed on a surface of the implantable medical device and a barrier membrane disposed on the drug-eluting membrane and inserting the implantable device into the patient. The drug-eluting membrane can comprise one or more therapeutic agents in a biologically stable or a non-bioerodible polymer matrix. The implantable device can further have a barrier membrane comprising a permeable polymer matrix disposed typically on the drug-eluting membrane that can control a rate of elution of the therapeutic agent from the drug-eluting membrane. In accordance with one or more embodiments, the invention can be characterized as providing a device. The device can be an implantable medical device which can comprise, for example, a body having a surface, a substance-eluting membrane as, for example, a drug-eluting membrane, disposed on at least a portion of the surface, and an elution rate-controlling layer or barrier membrane disposed on at least a portion of the substance-eluting membrane. The substance-eluting membrane can comprise one or more agents, including, for example, therapeutic agents or drugs, dispersed or infused in a matrix such as, but not limited to, a non-bioerodible polymer matrix. The rate-controlling layer or barrier membrane can comprise a permeable polymer matrix. In some cases, the membrane can have a thickness that provides a desired, predetermined or target transport or elution rate of the agent from the substance-eluting membrane to, for example, tissue or media surrounding or adjacent to the implantable medical device.

The invention can utilize, or be utilized in, a variety of devices, or components of devices, including, but not limited to, implantable medical devices such as stents, seals, sensors, transducers, catheters, probes, prosthetic devices, hip joints, bone implants such as screws, brackets and plates, intraocular devices, pace-makers, and needles. The invention can facilitate biological therapy by providing medicated devices such as, but not limited to implantable stents. Medicated stents can provide local administration of therapeutic substances at, i.e. focused or limited to, a diseased region, which may be advantageous over systemic administration techniques, which may produce global or system-wide adverse or even toxic effects. Local delivery can be a preferred method of treatment, providing smaller total levels of medication relative to systemic dosages, but concentrated at a specific site. Local delivery can thus produce fewer side effects and can achieve more favorable results. Thus, in accordance with one or more embodiments, the invention can be characterized as delivering or at least facilitating delivery of a substance, such as a drug or other therapeutic agent, to, preferably, a local, specific, or non-systemic region.

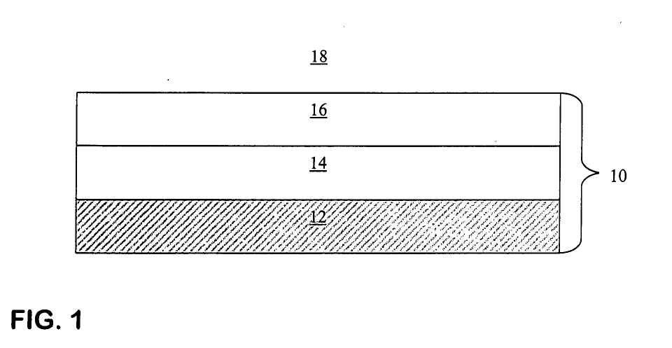

For example, FIG. 1 shows a portion of a device 10 having a body 12. Disposed on at least a portion of body 12 is a first membrane, which typically can be an eluting membrane 14, such as a drug-eluting membrane, containing a biologically active material such as a therapeutic agent. Disposed on at least a portion of membrane 14 is a transport rate-controlling membrane 16 having a thickness that controls the transport rate of the biologically active material or agent from membrane 14 to, for example, adjacent tissue 18. Typically transport rate-controlling membrane 16 comprises a material that is permeable or permits the transport of the elutable substance therethrough.

The elutable substance or agent can be any substance that is intended to be introduced to a medium, which can be biological tissue. The agent can be a therapeutic substance such as a drug or other biologically active material. Examples of agents include, but are not limited to, dexamethasone and derivatives thereof as well as paclitaxel and derivatives thereof. Thus, in accordance with one or more embodiments the invention can be characterized as providing parylene coatings as drug eluting membranes by incorporation of dexamethasone or a derivative thereof via solvent assisted swelling into the polymer membranes.

The coatings of the invention can comprise one or more polymeric materials. One or more of the polymeric materials can be substantially or at least partially particle-free. In some cases, the coating can comprise one or more membranes that are substantially homogeneous for at least a portion, region, or section thereof. As used herein, substantially particle-free refers to materials having little or only trace levels of particles at a level or concentration that does not affect the transport rate of the elutable substance through the substantially particle-free material.

In accordance with one or more preferred embodiments of the invention, the coatings can comprise one or more biologically compatible, or at least a biologically inert, materials. For example, the coatings can comprise biologically inert, stable, or non-bioerodible compatible materials that do not or at least have little or substantially no interaction with biological tissue. Preferably, the coatings of the invention can comprise one or more materials that do not produce any appreciable amounts of by-products upon exposure to living tissue. More preferably, the coatings do not produce any toxic substances by, for example, interaction or degradation, or at least, do not produce substances at toxic levels upon exposure to living tissue.

The coatings of the invention can comprise one or more sources of elutable or transportable agents or substances. The source or reservoir can comprise one or more membranes or layers, typically referred to as the eluting membrane or carrier membrane, having the one or more agents dispersed and/or stored therein. The amount of elutable substance or agent in the eluting membrane can be specified or predetermined to provide a total exposure or dosage. Thus, at least a portion or, in some cases, a substantial portion of the agent can be removed from the eluting membrane to provide an exposure amount of the agent. For example, the coatings of the invention can be disposed in an environment thereby exposing such, up to a predetermined total exposure dose. The total dose can be based on the initial available agent amount in the source or on the total elutable amount.

The eluting membrane can comprise a non-bioerodible polymer having a biologically active material dispersed therein. The method can further comprise an act of depositing a permeable membrane on the eluting membrane. The non-bioerodible polymer can comprise parylene or a derivative thereof. The permeable membrane can also comprise parylene or a derivative thereof. Thus, the invention can be characterized as providing a method of coating an implantable medical device comprising one or more acts of depositing a first polymer matrix comprising parylene (poly-para-xylylene, para-xylylene, or PPX) or a derivative thereof on a surface of the implantable medical device, infusing the first polymer matrix with a therapeutic agent, and depositing a second polymer matrix comprising parylene or a derivative thereof on the first polymer matrix. Further aspects of the invention involve one or more acts of depositing the eluting membrane can comprise one or more steps involving one or more parylene precursors. In some cases, the act of depositing the substance-eluting membrane can comprise infusing the biologically active material into the non-bioerodible polymer. The act of depositing the permeable membrane can comprise vaporizing one or more parylene precursors. Depositing the rate-controlling layer or permeable membrane can be performed so as to achieve a predetermined barrier membrane thickness. Preferably, the predetermined membrane thickness can provide or result in a device having a specific or target substance-elution rate into the environment surrounding the device. The predetermined membrane thickness can be between about 50 nm to about 5,000 nm, and typically, can be between about 50 nm to about 2,000 nm thick, preferably, between about 500 nm to about 2,000 nm thick. The elution rate can be from about one hour to about one year; preferably, from about one hour to about six months, more preferably from about one hour to about ninety days but in some cases, from about one day to about 30 days, depending on, among other considerations, the desired treatment or exposure parameters.

Infusing the biologically active material can comprise immersing the eluting membrane in a solution comprising the biologically active material. Infusing the first polymer matrix can also comprise immersing the first polymer matrix deposited on the implantable medical device in a solution having one or more therapeutic agents dissolved therein. The solution can comprise one or more solvents selected to promote mobilization of the one or more elutable agents into the eluting membrane or the first polymer matrix. For example, the solvent can be a non-aqueous liquid such as, for example, chloroform, methylethyl ketone, ethanol or even a supercritical fluid such as supercritical CO₂. The elutable substance can also be disposed in or on the substance-eluting membrane by deposition thereof on the membranes by, for example, spray coating, dip coating, or spin coating.

In some cases, the method can further be characterized as providing a controlled rate of deposition of the second polymer matrix to a predetermined thickness.

The device can, for example, comprise a stent, which can be used to provide a mechanical intervention but can also serve as a vehicle for providing biological therapy. As mechanical intervention, stents can act as scaffoldings that physically hold open and, if desired, to expand the wall of a passageway. Typically stents are capable of being compressed, so that they can be inserted through small cavities via catheters, and then expanded to a larger diameter when they are at the desired location.

The invention can be further characterized as being directed to one or more coatings or techniques for applying one or more coatings on a device, including, but not limited to, implantable medical devices such as stents. One or more coatings can have a plurality of layers including, for example, a first layer having one or more therapeutic agents or biologically active materials or compounds dispersed or infused therein. Preferably, the agents or compounds can diffuse from the first layer to a surrounding environment. The invention can also be characterized as infusing or otherwise promoting dispersion of one or more substances into a matrix, such as a polymer matrix. In other cases, the invention can be characterized as storing one or more substances in a polymeric material matrix. The invention can also be characterized as coatings having one or more agents or substances that can be eluted, diffused, or otherwise be removed therefrom to, for example, a surrounding or outside environment. In accordance with one or more embodiments, the invention can be characterized as providing one or more agents or substances that can undergo mass transport, such as by diffusion, through one or more barrier or elution rate controlling membranes to a target region. The one or more barrier membranes or layers can be constructed and arranged to achieve specific target transport rates of the agents or substances from one or more reservoirs or sources. Thus, in some cases, the invention can be characterized as delivering one or more substances, including, for example, therapeutic or biologically active substances in a controlled, uniform or at a constant dose.

The eluting membrane preferably comprises a material that can absorb or be infused with one or elutable or mobile agents or substances. Preferably, the eluting membrane can comprise a non-bioerodible material. For example, the eluting membrane can comprise a polymeric material including but not limited to parylene or a derivative thereof.

In some cases, a portion, region, or section of the eluting membrane can be loaded or infused with a first elutable agent and a second portion, region, or section can be loaded with a second elutable agent. In other cases, a first portion can be infused or comprise one or more elutable agents and a second portion, which need not necessarily be adjacent to or abut the first portion, and can comprise one or more elutable agents at a concentration that differs from the concentration of the eluting agents in the first portion.

The coatings of the invention can further comprise one or more rate-controlling membranes or layers, typically referred to as a permeable membrane, that can control or attenuate a rate of elution or transport of one or more of the agents from the one or more sources. The permeable membranes can further isolate from exposure, at least a portion of the device from its surrounding environment. The coating can also provide an intimal surface that promotes, or at least does not discourage, organic tissue growth on or adjacent to the device. Preferably, the coating can at least partially render or provide lubricity to the device. In some cases, the permeable membrane can also be infused with one or more substances that facilitate tissue growth or facilitate tissue repair or regeneration.

The membrane can comprise a permeable, and in some cases, porous material. For example, the membrane can comprise a polymeric material that is permeable or at least allows one or more of the agents to diffuse or transport therethrough. Preferably, one or more of the membranes can comprise a polymeric material that is substantially void-free. More preferably, one or more barrier membranes can comprise a non-hydrogel material.

The membrane can be deposited on at least a portion of a surface of the source or eluting membrane. Preferably, the membrane can be disposed on substantially all of an exposed surface of the eluting membrane. Thus, for example, the membrane can be deposited on one or both surfaces of one or more of the eluting membranes. The permeable barrier membrane can be disposed on a portion of the eluting membrane and allow control or at least defining a predetermined region through which the eluting substance can transport. Thus, in some cases, the membrane can comprise one or more regions or portion that are permeable to the elutable substance and one or more regions that are impermeable or at least are less permeable or provide a reduced or attenuated transport rate of the elutable substance.

In accordance with still further embodiments of the invention, the permeable membrane can have a thickness that is selected to control a rate of the transport or elution of the elutable substance. The membrane can comprise a permeable polymer that has a predetermined thickness so as to provide a predictable transport rate of one or more of the elutable substances, e.g. the biologically active agents, from the eluting membrane. For example, the permeable membrane can comprise parylene or a derivative thereof.

Parylene can be applied by utilizing a deposition chamber in a process typically referred to as vapor deposition polymerization. Such techniques typically involve the sublimation of a vapor that has been formed by heating dry, powdered, or liquid raw materials, one or more precursor materials, typically referred to as dimers. At room temperature in a vacuum, the vapor can convert to an inert polymer membrane on the substrate surface. Thus, the technique can be performed in a dry, non-solvent or solvent-free, and catalyst-free process. The precursor typically comprises pure, little, or no substantial amounts of foreign substances that could degrade medically-suitable surfaces. Preferably, the parylene membranes of the invention are pinhole-free and possess useful dielectric and barrier properties, per unit thickness, as well as exhibit chemical and biological inertness. Any one or more grades or variants of parylene compounds can be utilized in the systems, devices, and techniques of the invention. Examples of grades or types of parylene that can be utilized in accordance with one or more embodiments of the invention include HT-parylene, parylene C, parylene N, and even parylene D.

The extent or loading amount of the elutable agent in the eluting membrane can vary and can be tailored to achieve specific, predetermined total dosage or exposure amounts. For example, a total dosage can be prescribed and delivered to a target amount by introducing or utilizing units of coated devices having discrete units of elutable substances. However, in some cases, the loading amount may depend on the duration of exposure of the elutable agent so that a coated device can provide a target exposure dose during a period.

A technical advantage can be achieved by utilizing additional parylene cover coatings on top of the drug-eluting parylene coatings, which can provide a retarded release of one or more of the drugs. The invention can also provide nearly linear or controlled elution rates. Such rates typically correspond to the thickness of the parylene coating.

The function and advantages of these and other embodiments of the invention can be further understood from the examples below. The following examples illustrate the benefits and/or advantages of the one or more systems and techniques of the invention but do not exemplify the full scope of the invention.

### EXAMPLES

The following procedure was utilized for the examples. Membranes of parylene C and/or parylene N (detached from a glass substrate) were loaded with dexamethasone and dried in air. The parylene membranes were placed in about 20 ml of a buffer solution. The buffer solution (ethanol:buffer) consisted of about 60 vol % aqueous phosphate buffer (physiological buffer, pH about 7.41) and about 40 vol % ethanol (analytic grade) to mimic the conditions in the blood system.

Samples of the ethanol:buffer solution were taken at defined periods of time and evaluated to determine its dexamethasone concentration. The samples of the solution were measured by means of quantitative absorption-spectroscopy (UV/Vis).

### Example 1

Parylene N as well as parylene C were used as drug-loaded (here dexamethasone) substrate layers. The dexamethasone loading capacity of uncoated layers of parylene C was found to be higher than that of parylene N. The maximum total amount of dexamethasone can be related to the parylene C membrane thickness.

A loading solution was prepared by mixing about 15 mg dexamethasone in about 20 ml CHCl₃. To load the parylene C membranes, each were immersed in the loading solution for about one day. Each of the membranes had about equal exposed surface areas (about 37.5 mm²) but different membrane thickness (corresponding to mass of about 18 mg, about 28 mg, and about 91 mg). The dexamethasone-loaded parylene C membranes were then immersed in about 20 ml of an ethanol:buffer solution. Samples of the immersing solution were retrieved and analyzed to determine the dexamethasone concentration released thereinto.

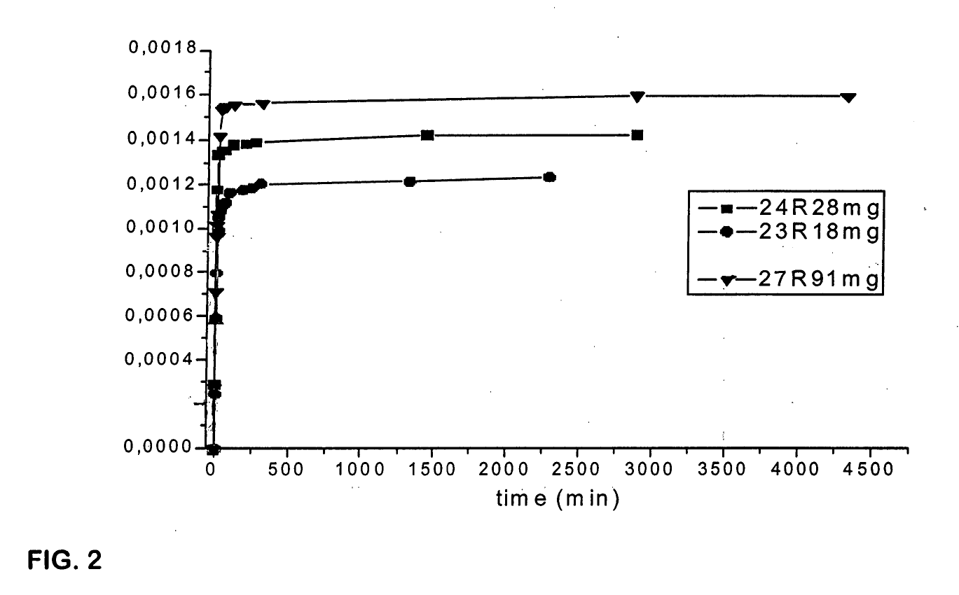

FIG. 2 is a graph showing the concentration of dexamethasone in the immersing solution as a function of time for parylene C membranes of different masses. The release of dexamethasone was quantified by UV/Vis spectroscopy techniques. The results presented in FIG. 2 show that the total amount of dexamethasone released typically increases with an increase in membrane thickness. This indicates that the drug is not only bound to the surface of the parylene membrane but also penetrates into the bulk of the membrane and furthermore can be released therefrom in use.

### Example 2

Test samples of parylene C membranes of substantially identical mass (about 90 mg) and substantially the same surface area (about 37.5 mm²) were loaded in solutions of CHCl₃ (about 20 ml)/dexamethasone (about 15 mg) for different periods, ranging from about one day to about 22 days. The dexamethasone-loaded samples were then immersed in an ethanol:buffer solution; from which, samples were periodically retrieved and analyzed to determine dexamethasone concentration by UV/Vis spectroscopy techniques.

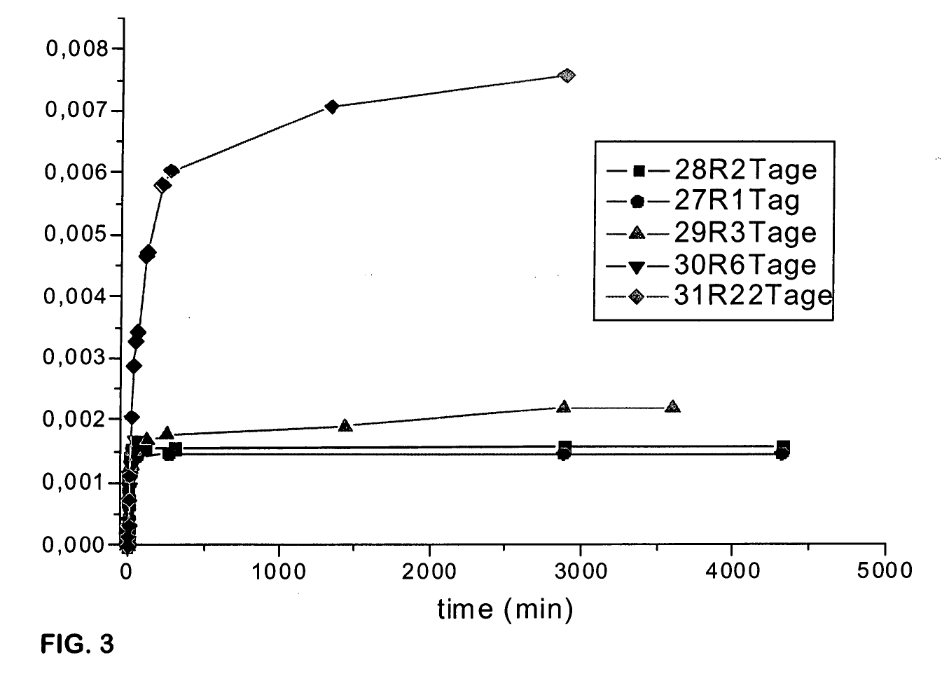

FIG. 3 shows the effect of dexamethasone drug loading uptake time on parylene C membranes of substantially the same mass and substantially the same surface area. The results show that extending drug loading time resulted in a significant increase of drug loading into the parylene C membrane. Thus, the amount of drug loading can be controlled by regulating the total loading period. Due to experimental reasons, the release experiment of run 31 (about 22 days) was stopped but clearly shows that the release of dexamethasone was still in progress. In particular, the data shows that more than about 0.0075 mg of dexamethasone can be loaded into about 90 mg of a parylene membrane (about 37.5 mm²) and be controllably released therefrom to provide a total delivered dosage. The data also shows that significant release amounts of a drug can be expected for a period of more than about 50 hours.

### Example 3

Release of dexamethasone from parylene membranes can result in burst release under physiological conditions, which may be desired but for therapeutic reasons, a linear release or combination of burst release and linear release may be advantageous. This example studies the controlled release of dexamethasone from parylene N loaded by dexamethsone from CHCl₃ solution and covered by additional parylene N layer of different thicknesses was evaluated tested. Parylene N membrane samples were prepared and loaded with dexamethasone as substantially described in the previous examples. The dexamethasone-loaded parylene N samples were immersed in an ethanol:buffer solution; from which, samples were periodically retrieved and analyzed as also described in the previous examples.

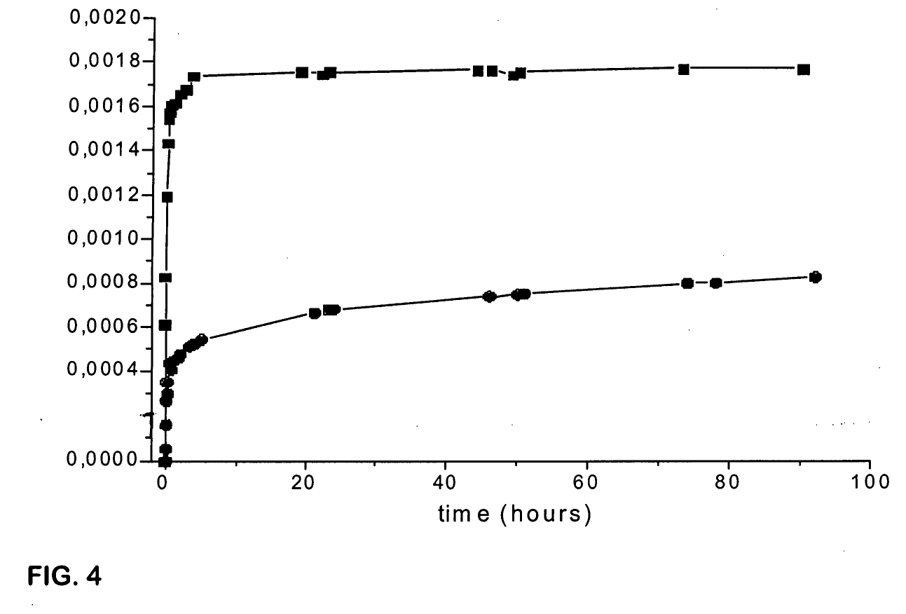

FIG. 4 is a graph showing a comparison of the release of dexamethasone from an uncoated dexamethasone-loaded parylene N membrane and a parylene N coated dexamethasone-loaded parylene N membrane. The uncoated parylene N membrane (“control-1”) had a mass of about a 29 mg and a surface area of about 37.5 mm². It was loaded by immersion in a dexamethasone/CHCl₃ solution for about one day. The coated membrane (“sandwich-1”) was comprised of a dexamethasone-loaded layer of parylene N and a coating also comprising parylene N. The coating was about 800 nm thick. The dexamethasone-loaded layer had a mass of about 32 mg and an effective exposed surface area of about 37.5 mm². This dexamethasone-loaded layer was also loaded by immersion in the dexamethasone/CHCl₃ solution for about one day. Both samples were immersed in ethanol:buffer solutions from which samples were retrieved and analyzed.

The results presented in FIG. 4 shows that the release profile of the coated sample (sandwich-1) differs from the release profile of the control-1 sample. Significantly, the data shows that the drug release rate can be controlled or regulated to provide a controlled, graduated dexamethasone release.

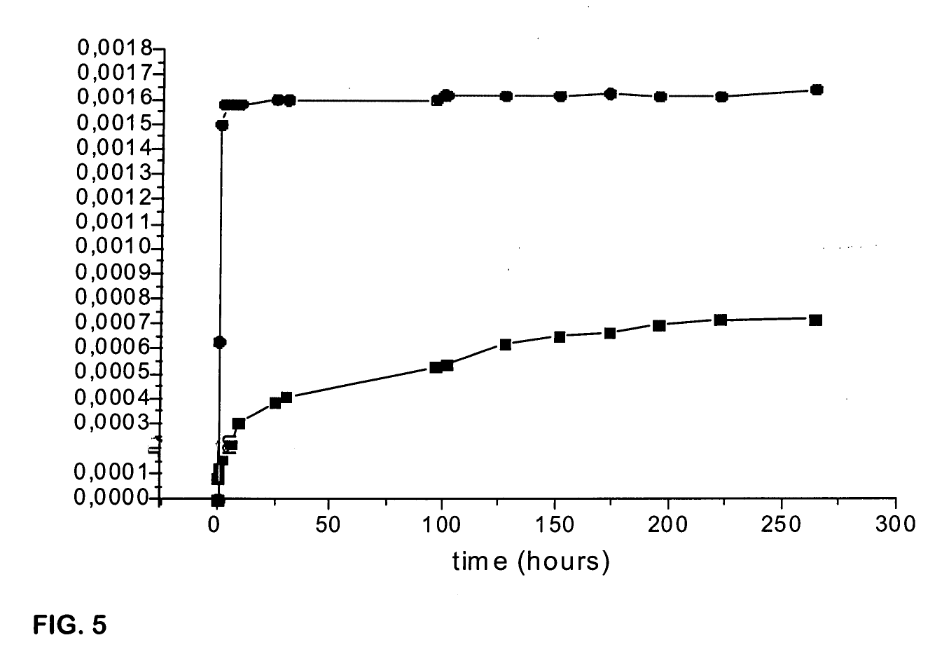

To further study the utility for controlling the drug release rate, a second coated sample was prepared as substantially described above. This coated sample (“sandwich-2”) was comprised of a parylene N coating which was about 1700 nm thick on a dexamethasone-loaded parylene N layer that had a mass of about 18 mg and an effective surface area of about 37.5 mm². The coated sample (sandwich-2) was loaded by immersion in a dexamethasone/chloroform solution for about one day. A second control sample (“control-2”) was also utilized which was an uncoated, dexamethasone-loaded parylene N sample with a mass of about 18 mg and an effective surface area of about 37.5 mm². The control-2 sample was also loaded by immersion in a dexamethasone solution for about one day. The coated and uncoated samples were each immersed in an ethanol:buffer solution as substantially described above. Samples of the solutions solutions were periodically retrieved and analyzed also as substantially described above. The measured dexamethasone concentrations were collated and are presented in FIG. 5.

FIG. 5 is a graph showing a comparison of the release of dexamethasone from the uncoated dexamethasone-loaded parylene N membrane and the parylene N-coated (about 1700 nm membrane thick) dexamethasone-loaded parylene N membrane (about 18 mg parylene N, surface area of about 37.5 mm², loading period of about one day).

Permeable membrane coatings of parylene N, with thicknesses of about 800 nm and about 1700 nm were tested with different amounts of dexamethasone-loaded parylene N membranes. The results show that the release of dexamethasone tends to an extended linear relationship between drug release versus time with thicker the membranes. The results show that coatings and techniques of the invention can be utilized to controllably deliver an agent without a burst release. In particular, as shown in FIGS. 4 and 5, unloaded cover membranes of parylene N attenuated the release rate of dexamethasone from the loaded layer. Burst release of the dexamethasone from the eluting membrane or layer was also reduced, if not eliminated. It is believed that diffusion controlled release continued even after about 300 hours. That is, the results show that the release of a target or active substance from an eluting membrane or layer can be controlled by selecting and applying a layer, typically permeable to the eluting substance, on the eluting membrane.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. For example, the use of several agents in one or a plurality of membranes arranged in layers or in adjacent regions is contemplated by one or more embodiments of the invention. Although many of the examples presented herein involve specific combinations of method acts or system elements, it should be understood that those acts and those elements may be combined in other ways to accomplish the same objectives. For example, the step or act of infusing an eluting membrane with one or more active components can be performed utilizing one or more various techniques of exposing one or more eluting membranes to the one or more active components. Acts, elements and features discussed only in connection with one embodiment are not intended to be excluded from a similar role in other embodiments. It is to be appreciated that various alterations, modifications, and improvements can readily occur to those skilled in the art and that such alterations, modifications, and improvements are intended to be part of the disclosure and within the spirit and scope of the invention. Moreover, it should also be appreciated that the invention is directed to each feature, system, subsystem, or technique described herein and any combination of two or more features, systems, subsystems, or techniques described herein and any combination of two or more features, systems, subsystems, and/or methods, if such features, systems, subsystems, and techniques are not mutually inconsistent, is considered to be within the scope of the invention as embodied in the claims.

Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Further, as used herein, “plurality” means two or more. The invention can also be directed to a set including one or more devices, coated devices, as well as kits including devices, coated devices with or without the elutable substance dispersed therein along with one or more solutions of the elutable substance for dispersion or infusion into the devices or coated devices. As used herein, a “set” of items may include one or more of such items.

Those skilled in the art should appreciate that the parameters and configurations described herein are exemplary and that actual parameters and/or configurations will depend on the specific application in which the systems and techniques of the invention are used. Those skilled in the art should recognize or be able to ascertain, using no more than routine experimentation, equivalents to the specific embodiments of the invention. It is therefore to be understood that the embodiments described herein are presented by way of example only and that, within the scope of the appended claims and equivalents thereto; the invention may be practiced otherwise than as specifically described.

What is claimed is:

1. A method of coating a medical device comprising:
depositing an eluting membrane on a surface of the implantable medical device, the eluting membrane comprising a non-bioerodible polymer having a biologically active material dispersed therein; and
depositing a permeable membrane comprising a permeable polymer on the eluting membrane.

2. The method of claim 1, wherein the non-bioerodible polymer comprises parylene or a derivative thereof.

3. The method of claim 2, wherein the non-bioerodible polymer comprises a parylene copolymer.

4. The method of claim 2, wherein the permeable polymer comprises parylene or a derivative thereof.

5. The method of claim 4, wherein the permeable polymer comprises a parylene copolymer.

6. The method of claim 4, wherein depositing the eluting membrane comprises vaporizing a parylene precursor.

7. The method of claim 6, wherein depositing the eluting membrane comprises infusing the biologically active material into the non-bioerodible polymer.

8. The method of claim 7, wherein depositing the eluting membrane comprises co-deposition of the biologically active material and the non-bioerodible polymer.

9. The method of claim 7, wherein infusing the biologically active material comprises immersing the eluting membrane in a solution comprising the biologically active material.

10. The method of claim 9, wherein depositing the permeable membrane comprises vaporizing a parylene precursor.

11. The method of claim 10, wherein the biologically active material comprises dexamethasone or a derivative thereof.

12. The method of claim 10, wherein the biologically active material comprises paclitaxel or a derivative thereof.

13. The method of claim 1, wherein depositing the permeable membrane is performed to achieve a predetermined permeable membrane thickness.

14. The method of claim 13, wherein the predetermined permeable membrane thickness provides a target drug-elution rate.

15. The method of claim 14, wherein the predetermined permeable membrane thickness is between about 50 nm and about 5000 nm.

16. The method of claim 15, wherein the thickness is between about 500 nm and about 2000 nm.

17. The method of claim 15, wherein the thickness is between about 50 nm and about 2000 nm.

18. A method of coating an implantable medical device comprising:
depositing a first polymer matrix comprising parylene or a derivative thereof on a surface of the implantable medical device;
infusing the first polymer matrix with a therapeutic agent; and
depositing a second polymer matrix comprising parylene or a derivative thereof on the first polymer matrix.

19. The method of claim 18, wherein infusing the first polymeric matrix comprises immersing the first polymer matrix in a solution having the therapeutic agent dissolved therein.

20. The method of claim 19, wherein the solution comprises a solvent selected to promote mobilization of the therapeutic agent into the first polymer matrix.

21. The method of claim 20, wherein the solvent is non-aqueous.

22. The method of claim 20, wherein the solvent is aqueous.

23. The method of claim 20, wherein the solvent comprises a supercritical substance.

24. The method of claim 23, wherein the solvent comprises supercritical CO₂.

25. The method of claim 18, further comprising controlling a rate of deposition of the second polymer matrix to a predetermined thickness.

26. A method of administering a drug to a patient comprising:
providing a medical device comprising a drug-eluting membrane disposed on a surface of the medical device and a permeable membrane disposed on the drug-eluting membrane, the drug-eluting membrane comprising a drug infused in a non-bioerodible polymer and the permeable membrane comprising a polymer; and
implanting the medical device in the patient.

27. The method of claim 26, wherein the drug is dexamethasone or a derivative thereof.

28. The method of claim 26, wherein the drug is paclitaxel or a derivative thereof.

29. The method of claim 26, wherein the amount of the drug in the drug-eluting membrane provides a target drug dosage during a predetermined period.

30. A method of coating an medical device comprising:
depositing a first polymer matrix comprising parylene or a derivative thereof on a surface of the medical device; and
infusing the first polymer matrix with a therapeutic agent.

31. The method of claim 30, further comprising depositing a second polymer matrix comprising parylene or a derivative thereof on the first polymer matrix.

32. A method of facilitating delivery of a therapeutic agent comprising:
providing a medical device comprising an eluting membrane disposed on a surface of the medical device and a permeable membrane disposed on the eluting membrane, the eluting membrane comprising a therapeutic agent infused in a non-bioerodible polymer, and the permeable membrane comprising a polymer.

33. The method of claim 32, wherein the permeable membrane has a thickness that provides a target transport rate of the therapeutic agent from the eluting membrane.

34. The method of claim 33, wherein the predetermined permeable membrane thickness is between about 50 nm and about 5000 nm.

35. The method of claim 34, wherein the thickness is between about 500 nm and about 2000 nm.

36. The method of claim 32, wherein the non-bioerodible polymer comprises parylene or a derivative thereof.

37. The method of claim 37, wherein the permeable polymer comprises parylene or a derivative thereof.

38. An implantable medical device comprising:
a body having a surface;
a drug-eluting membrane disposed on the surface, the drug-eluting membrane comprising a therapeutic agent infused in a non-bioerodible polymer; and
a permeable membrane disposed on the drug-eluting membrane, the barrier membrane comprising a polymer.

39. The implantable medical device of claim 38, wherein the permeable membrane has a thickness that provides a predetermined transport rate of the therapeutic agent from the drug-eluting membrane.

40. The implantable medical device of claim 39, wherein the thickness is between about 50 nm and about 5000 nm.

41. The implantable medical device of claim 40, wherein the thickness is between about 500 nm and about 2000 nm.

42. The implantable medical device of claim 38, wherein the drug-eluting membrane comprises parylene or a derivative thereof.

43. The implantable medical device of claim 42, wherein the permeable membrane comprises parylene or a derivative thereof.

44. The implantable medical device of claim 38, wherein the therapeutic agent comprises dexamethasone or a derivative thereof.

45. The implantable medical device of claim 38, wherein the therapeutic agent comprises paclitaxel or a derivative thereof.
